(19) United States
(12) Patent Application Publication
(10) Pub. No.: US 2007/0086972 A1
(43) Pub. Date: Apr. 19, 2007

(54) HAIR GROWTH COMPOSITIONS AND METHODS FOR TREATING HAIR LOSS OR RELATED CONDITIONS

(76) Inventor: Jacob Birnbaum, New York, NY (US)

Correspondence Address: Jacob Birnbaum, Apt 8P, 305 East 40th Street, New York, NY, 10016, US

(21) Appl. No.: 11/530,501

(22) Filed: Sep. 11, 2006

Related U.S. Application Data
(60) Provisional application No. 60/716,156, filed on Sep. 12, 2005.
(60) Provisional application No. 60/784,291, filed on Mar. 21, 2006.

(51) Int. Cl. A61K 35/12; A61K 8/97

(52) U.S. Cl. 424/74; 424/93.7

(57) ABSTRACT

The present invention relates to hair growth compositions as well as to methods for treating hair loss or related conditions.

This application claims priority from U.S. provisional application 60/716,156, filed Sep. 12, 2005, and U.S. provisional application 60/784,291 filed Mar. 21, 2006, both of which are incorporated herein by reference.

### BACKGROUND OF THE INVENTION

In humans, each hair follicle goes through repeated cyclical periods of growth including an active growth stage (anagen), which can persist for approximately 2 to 6 years; a transition phase (catagen), which lasts for only a week or two; and a resting period (telogen), which lasts 3 to 4 months. The hair is shed at the end of the telogen phase, and a new hair is grown as the cycle repeats. In the human scalp, which contains approximately 100,000 hair follicles, normally about 86% are in anagen, 1% are in catagen and 13% are in telogen. Therefore, in a normal human adult, approximately 100 hairs are shed from the scalp per day.

Excessive hair loss, or alopecia, may be classified as being one of two types, non-scarring alopecia and scarring alopecia, and can be caused by a wide variety of factors. For example, non-scarring alopecia has been attributed to genetics and advanced age; administration of drugs such as anti-cancer chemotherapeutic drugs and contraceptives; topical use of chemical treatments, such as hair dyes, permanent wave solutions, and straighteners; diseases, such as leprosy or syphilis; illness; allergy; and hair follicle infection. Scarring alopecia may be a consequence of burns (accidental or post surgical from cryosurgery or laser surgery) or trauma, which often causes destruction of follicles.

The most common type of human hair loss is androgenetic alopecia (also known as androgenic alopecia), which is a non-scarring hair loss of telogen hairs caused by an excessive androgen effect in genetically susceptible men and women. Androgens trigger the miniaturization or atrophy of terminal follicles that normally produce thick scalp hair and transforms them into vellus-like follicles, eventually yielding fine, downy hair that is barely perceptible. Androgenetic alopecia is expressed in males as baldness of the vertex of the scalp and is commonly referred to as male pattern baldness. In females, androgenetic alopecia appears as diffuse hair loss or thinning of the frontoparietal areas. As alopecia progresses with age, hairs in these predisposed areas miniaturize and appear to change from terminal hairs to resemble vellus hairs. In addition, as androgenetic alopecia continues, the number of hairs in the active growth anagen phase decreases while there is an increase the number of hairs in the telogen phase.

Androgenetic alopecia, which is sometimes referred to as “common baldness” or “male pattern baldness,” independent of its causes, is the cutaneous aping of a particular zone (i.e., the scalp). Androgenetic alopecia can be defined, on one hand, as atrophy, sclerosis or minaturization of the hair follicles. On the other hand, androgenetic alopecia can be defined as a progressive shortening of the average duration of the anagen stage, which results in vellus hair prior to complete disappearance.

Hair loss is an extremely common condition among healthy adult males, and also occurs frequently in adult females. In fact, some degree of alopecia on the vertex from puberty onwards is thought to be a universal phenomenon in both men and women (R. P. R. Dawber (1987) Dermatologica 175:23-28). Alopecia is also frequently observed in both pre- and post-pubertal patients as a side-effect of anti-cancer chemotherapy, (A. M. Hussein, et al. (1990) Science 249:1564-1566, B. W. Cline, (1984) Cancer Nursing 7:221-228; A. F. Hood (1986) Med. Clin. North Am. 70:187-209).

Despite the widespread occurrence of androgenetic alopecia, the need for prevention and therapy still exists. The lack of a proven and effective treatment for androgenetic alopecia has caused many afflicted individuals to adopt the practice of wearing a wig or toupee. Another extreme measure used to combat androgenetic alopecia, hair transplant surgery, is not available as an option in many cases (i.e., following chemotherapy) and offers, at best, only a partial remedy. At the same, the latter treatment suffers from a number of disadvantages, including the need for surgery.

A common non-surgical treatment for stimulating hair growth is minoxidil (The Upjohn Company, Kalamazoo, Mich.). A solution of minoxidil as active ingredient is known as Rogaine.R™. As stated in the Rogaine.R™. Patient Information Booklet (The Upjohn Company, Kalamazoo, Mich., revised June, 1992) minoxidil is a vasodilatory drug which has serious side-effects when administered orally for the treatment of hypertension. At the same time, topical application of minoxidil for the treatment of androgenetic alopecia is only partially effective and suffers from a number of disadvantages. For example, minoxidil is only recommended for treatment of male pattern alopecia of the vertex (i.e., frontal recession), has to be applied twice daily for at least four months, and requires a normal scalp with no local abrasions, dermatitis or sunburn—conditions that can increase absorption into the blood stream and the concomitant risk of side-effects. Further, minoxidil is of limited effectiveness. For example, there is no significant increase in terminal hair growth between minoxidil and placebo treatment groups after four months of treatment (refer to the Rogaine.R™. Patient Information Booklet, The Upjohn Company, Kalamazoo, Mich., revised June, 1992). In patients who do respond to minoxidil treatment, the new hair is likely to be shed within a few months after stopping treatment.

Androgens are responsible for many physiological functions in both males and females. Androgen action is mediated by specific intracellular hormone receptors expressed in androgen responsive cells. Testosterone, the major circulating androgen, is secreted by Leydig cells of the testes under the stimulation of pituitary-derived luteinizing hormone (LH). However, reduction of the 4, 5 double bond of testosterone to dihydrotestosterone (DHT) is required in some target tissues, such as prostate and skin, for androgen action. Steroid 5.alpha.-reductases in target tissues catalyze conversion of testosterone to DHT.

The requirement for DHT to act as an agonist in these target tissues has been highlighted by studies of steroid 5.alpha.-reductase deficient individuals who have vestigial prostate glands and do not suffer from male pattern baldness (see McGinley. J. et al., The New England J. of Medicine, 300, 1233 (1979)). Thus, inhibition of the conversion of testosterone to DHT in these target tissues is anticipated to be useful in the treatment of a variety of androgen responsive diseases (i.e., benign prostatic hyperplasia, prostate cancer, acne, male pattern baldness and hirsutism).

Additionally, it has been discovered that two isozymes of 5.alpha.-reductase exist in humans that differ in their tissue distribution, affinity for testosterone, pH profile and sensitivity to inhibitors (see Russell, D. W. et al., J. Clin. Invest., 89, 293 (1992); Russell, D. W. et al., Nature, 354, 159 (1991)). The steroid 5.alpha.-reductase deficient individuals studied by Imperato-McGinley are deficient in the type 2,5.alpha.-reductase enzyme (Russell, D. W. et al., J. Clin. Invest., 90, 799 (1992); Russell, D. W. et al., New England J. Med., 327, 1216 (1992)), which is the predominant isozyme present in the prostate, while the type 1 isozyme is predominant in the skin. The relative value of isozyme specific and dual inhibitors of the two isozymes of 5.alpha.-reductase will depend upon the type of disease treated (benign prostatic hyperplasia, prostate cancer, acne, male pattern baldness or hirsutism) as well as the stage of the disease (prevention versus treatment) and the anticipated side-effects in the intended patients (for example treatment of acne vulgaris in pubescent males).

Because of their valuable therapeutic potential, testosterone 5.alpha.-reductase inhibitors have been the subject of active research worldwide. For example, see: Hsia, S. and Voight, W., J. Invest. Derm., 62, 224 (1973); Robaire, B. et al., J. Steroid Biochem., 8, 307 (1977); Petrow, V. et al., Steroids, 38, 121 (1981); Liang, T. et al. J. Steroid Biochem., 19, 385 (1983); Holt, D. et al., J. Med. Chem., 33, 937 (1990): U.S. Pat. No. 4,377,584, U.S. Pat. No. 4,760,071 and U.S. Pat. No. 5,017,568. Several notable 5.alpha.-reductase inhibitors are: (1) finasteride, (marketed under the trademark, Proscar); (2) SKF-105657; (3) cyproterone acetate, (4) duasteride (marketed under the trademark, Avodart).

For example, Finasteride (17.beta.-(N-tert-butylcarbamoyl)-4-aza-5.alpha.-androst-1-ene-3-one), which is marketed by Merck & Co., Inc. under the trade name PROSCAR.R™., is an inhibitor of 5.alpha.-reductase 2 and is known to be useful for the treatment of hyperandrogenetic conditions. See e.g., U.S. Pat. No. 4,760,071. Finasteride is currently marketed in the United States and worldwide for the treatment of benign prostatic hyperplasia. Finasteride's utility in the treatment of androgenetic alopecia is also disclosed in the following documents: EP 0 285,382, published Oct. 5, 1988; EP 0 285,383, published Oct. 5, 1988; Canadian Patent no. 1,302,277; and Canadian Patent no. 1,302,276.

Androgens are the most obvious regulators of human hair growth in both sexes. Androgens have paradoxically contrasting effects on follicles depending on their location in the body. Androgens stimulate hair growth in many locations (i.e., beard, axilla) while inhibiting scalp hair growth in genetically predisposed individuals. Androgens act on the hair follicles via the dermal papilla, presumably by altering the production of regulatory factors effecting the dermal papilla cells. Cultured dermal papilla cells secrete soluble, proteinaceous factors which are mitogenic for other dermal papilla cells, outer root sheath cells, epidermal keratinocytes and endothelial cells. Androgen sensitive cells from beard or balding scalp reflect their in vivo androgenetic responses by responding to testosterone, by increasing (i.e., beard) or decreasing (i.e., balding) their mitogenic ability.

The present invention relates to improvements in treating hair loss and/or means for causing hair growth/regrowth (and related conditions) stemming from exposure to anti-androgenetic agents.

### SUMMARY OF THE INVENTION

It is an object of the present invention to provide a hair growth composition comprising at least one anti-androgenetic agent and at least one blood-sugar modulating agent.

It is also an object of the present invention to provide a hair growth composition comprising at least one anti-androgenetic agent and at least one agent that can normalize at least one of a subject's body clocks or circadian rhythms.

It is also an object of the present invention to provide a method for the treatment of hair loss comprising providing to a living being an effective amount of any of the hair growth compositions described herein.

Anti-androgenetic agents are agents that can prevent, slow down, or stop the atrophy, sclerosis or minaturization of the hair follicles. Anti-androgenetic agents can also prevent or retard the progressive shortening of the average duration of the anagen stage, which results in vellus hair prior to complete disappearance. Examples of anti-androgenetic are Testosterone 5.alpha.-reductase inhibitors such as finasteride and duasteride and/or mixtures thereof. Other anti-androgenetic agents nonexclusively include: SKL-105657, Estrogen, Cyproterone acetate, spironolactor, flutamide, minoxidil, RU58841, 6-(1-piperdinyl)-2,4-pyrimidinediamine-3-oxide, N′-cyano-N-(tert-pentyl)-N′-3-pyridinyl-guanidine, retinoids and derivatives thereof, ketoconazole, elubiol or mixtures thereof, and mixtures of any of the above thereof.

Anti-androgenetic agents are now commonly used by people around the world in their attempts to regrow hair. However, despite the role that anti-androgenetic agents can serve in slowing down or stopping hair loss—or even growing or regrowing hair—many people stop using them after some period of time because they often find that the benefits do not outweigh many of their common and harsh side-effects.

For example, products containing minoxidil, when applied to the skin, cause scalp dryness, itchiness, irritation, and/or dandruff. After applying minoxidil, many users wind up scratching their scalps more frequently than they ordinarily would—in an attempt to relieve the discomfort caused by the minoxidil. As a result, many of these users wind up “scratching out” hair that they would have left alone if they had never applied minoxidil in the first place.

As another example, users of testosterone 5.alpha.-reductase inhibitors regularly experience severe sexual side-effects such as reduced libido, difficulty reaching ejaculation/orgasm, and/or impotence. Other potential side-effects include gynecomastia, breast tenderness, and/or breast enlargement. Additionally, some testosterone 5.alpha.-reductase inhibitors such as duasteride have relatively long half-lives in the bodies of humans so their side-effects can take a considerable amount of time to go away.

Users often find that the slight improvements that they see from exposing themselves to anti-androgenetic agents do not outweigh the totality of side-effects that they experience. As a result, many users discontinue exposing themselves to anti-androgenetic agents.

To address these problems, one aspect of the present invention mitigates the side-effects caused by exposure to anti-androgenetic agents—thereby leading users to continue taking anti-androgenetic agents to treat hair loss problems. Moreover, the present invention can also lead users to become more comfortable taking larger doses of anti-androgenetic agents. As a result, users can experience hair growth activity that can be more effective and/or less dangerous in the long run.

For example, users can reduce the irritation in the scalp area caused by the application of topical ointments containing minoxidil by employing a shampoo containing anti-fungal or anti-dandruff ingredients such as coal tar. In doing so, users will be more comfortable continuing to use minoxidil.

As an additional example, nutrients such as allicin and chemicals derived from the allium family of vegetables contain some of the most powerful antioxidants known, so these substances can help combat some of the long-term harmful effects of anti-androgenetic agents upon the skin of living beings. Moreover, sprouts such as vegetable sprouts (e.g. broccoli sprouts), grain sprouts (e.g. wheat sprouts), bean sprouts (e.g. mung bean sprouts), and herbal sprouts (e.g. mustard sprouts) contain remarkably high concentrations of antioxidants, so they can be useful in combating anti-androgenetic agents' harmful effects as well.

Another aspect of the present invention enhances the efficacy of various anti-androgenetic agents in the bodies of living beings.

For example, activities assisting in the relaxation of the neck, head, and scalp area may reduce scalp laxity over time and can facilitate in bringing greater blood-flow to hair follicles. This is important because hair follicles that do not receive an adequate blood supply will wither. Acts such as jaw-grinding (e.g. bruxism) can contribute to tension in the neck, head, and scalp area as well, so wearing a bite plate to prevent jaw-grinding can indirectly lead to greater blood flow to hair follicles as well. Vasodilators such as capsaicin can also directly lead to greater blood flow to hair follicles.

Adequate hydration through the regular consumption of water is essential for the optimal functioning of all organ systems including the maintenance of healthy skin.

Certain vitamins and nutrients such as lycopene and isoflavones (directly or by way of eating tomatoes or tofu) and/or the regular consumption of dairy products and/or increasing folate intake (directly or through eating foods such as spinach or fortified cereal) and/or exposure to sunlight, and/or ingesting adequate amounts of Vitamin D, and/or getting enough sleep appear to affect the rate of production of DHT in the bodies of living beings and the overall balance of hormones.

Nutrients such as omega-3 fatty acids—found in cold-water fish such as salmon and mackerel, nuts such as walnuts, and seeds such as flaxseed and pumpkin seeds—have been found to have numerous positive health effects ranging from controlling blood pressure to reducing depression. Several studies have suggested that omega-3 fatty acids can help delay hair loss or contribute to hair growth.

Pesticides, herbicides, and related chemicals can cause hair loss in the bodies of living beings. By consuming bioengineered foods or foods that have been certified as organic, users can reduce or eliminate the likelihood that such chemicals will contribute to hair loss.

Engaging in addictive behaviors can cause sudden and dramatic fluctuations in hormonal levels. Controlling these addictive behaviors by abstaining or withdrawing from the sources of the addiction and/or undergoing treatments for the addiction can affect the rate of production of DHT and the overall balance of hormones in the bodies of subjects.

Subjects' body clocks and circadian rhythms are directly related to their overall balance of hormones as well—so these clocks and/or rhythms should be normalized or regularized to some extent or as much as possible.

Allergens such as dust mites and their waste products can irritate the scalp of subjects during sleep. As a result, sleeping upon one or more pillows composed of one ore more anti-allergenic materials such as silk can mitigate or eliminate such irritation and further maintain the health of cells in the scalp area.

Additionally, reducing blood-sugar levels or keeping levels of blood sugar relatively constant through measures such as caloric restriction or reduction, or the eating of frequent but smaller-than-regular meals, has been shown to improve the health and luster of the hair of living beings in controlled laboratory settings. Moreover, a study reported in Lancet on Sep. 30, 2000 (356:1165-1166) suggested that insulin resistance could be a pathophysiological mechanism or promoting factor in early androgenic hair loss. Addressing insulin resistance or other blood-sugar issues through blood-sugar modulating agents or blood-sugar modulating activities can therefore incidentally or directly treat hair loss as well.

### DETAILED DESCRIPTION OF THE INVENTION

The present invention provides a hair growth composition comprising at least one anti-androgenetic agent and at least one blood-sugar modulating agent.

The act of exposing an individual to any of the substances, agents, or compounds in the present invention is an act that one or more individuals can do on their own behalves or on behalf of one or more others. Exposure to the substances or agents mentioned in this patent can be accomplished or aided through means nonexclusively including eating, chewing an article such as a gum, ingesting, consuming, drinking, eating, injecting, inhaling, snorting, swallowing, smoking, or topical application in the form of a patch, ointment, or lotion, laser treatments, light treatments, sound treatments, ultrasound treatments, and/or mixtures thereof.

Blood-sugar modulating agents are agents that can modulate, regulate, stabilize, or reduce a subject's blood sugar in the short-term or over a period of time and/or narrow the fluctuations in a subject's blood sugar in the short-term or over a period of time and/or improve the absorption of sugar and other nutrients. Blood-sugar modulating agents nonexclusively include substances that are used to treat diabetes or other blood-sugar disorders, substances that increase insulin sensitivity (e.g. increasing the sensitivity of muscle and fat cells to insulin), increase the number of insulin receptors, decrease glucose production by the liver, reduce gluconeogenesis (or the formation of new glucose), reduce glycogenolysis (or the breakdown of glycogen to glucose), interfere with the breakdown of complex carbohydrates and/or delay the absorption of glucose or other monosaccharides, deal with hyperinsulinemia or insulin resistance, antihyperglycemic agents, insulin augmenting agents, insulin-assisting agents, substances that lower insulin and blood sugar, substances that can turn on/off TORC switches or the genes necessary for gluconeogenesis in users' bodies, and/or combinations thereof.

Specific blood-sugar modulating agents nonexclusively include high concentrations of fiber, medications or substances nonexclusively including Insulin, Sulfonylureas, Biguanides, Meglitinides, Prandial glucose regulators, Alpha-glucosidase Inhibitors, Thiazolidinediones, Glitazones, Acarbose, aspirin, hormones (naturally occurring in humans and/or animals or synthetic versions), Byetta, Symlin, other agents used in the treatment or management of diabetes or blood-sugar disorders, and/or mixtures thereof.

Insulin includes all types of human insulin and natural (‘animal’) insulin. It can be obtained through means and sources nonexclusively including pancreatic or other tissue from humans or animals, through recombinant DNA technology, through genetic engineering, and/or combinations thereof.

Sulfonylureas are a class of drugs used to treat diabetes. They work by stimulating the pancreas to release more insulin from the beta cells. Secondary effects may include increasing insulin sensitivity, increasing the number of insulin receptors, and decreasing glucose production by the liver.

Biguanides are a class of drugs used to treat diabetes. Their mechanism of action is to decrease glucose production in the liver primarily by reducing gluconeogenesis and by reducing glycogenolysis. They can also have secondary effects of enabling glucose transport to occur in skeletal and fat tissue, and may decrease gastrointestinal absorption of glucose.

Thiazolidinediones are a class of drugs used to treat diabetes. They work by increasing the sensitivity of muscle and fat cells to insulin. They activate peroxisome proliferator-activated receptor gamma (PPAR gamma), which leads to a reduction in insulin resistance.

Alpha-glucosidase inhibitors (or glucosidase inhibitors) are a class of drugs used to treat diabetes. They inhibit enzymes in the brush border of the small intestines and pancreatic alpha lipase. They are effective in lowering postprandial hyperglycemia. By inhibiting the alpha-glucosidase enzymes, they interfere with the breakdown of complex carbohydrates and delay the absorption of glucose and other monosaccharides.

Meglitinides are a class of drugs used to treat diabetes. Their action is glucose-dependent and diminishes as blood glucose levels decrease. They can be used to lower postmeal blood glucose elevations

Specific blood-sugar modulating agents further nonexclusively include nutrients, herbs, spices, botanicals and/or botanical extracts nonexclusively including aconite, adenophora, alisma, amalaki churna, anemarrhena, asparagus root, astragalus, atractylodes, banaba plant, benincasa, bitter gourd, bitter melon, bilva, carthamus, chandraprabhavati, chromium, chrysanthemum, cinnamon, clerodendron, codonopsis, coix, coptis, cornus, corn silk, cumin, cuscuta, cyperus, Dakhni Mirch, dandelion, dendrobium, dioscorea, elecampane, epimedium, eriocaulon, fenugreek, fo-ti, fumar, ginseng, glehnia, glucomannan, gymnema, gypsum, ho-shou-wu, hoelen, jambu, jamun-ki-guthali, juniper berries, konjac, laminaria, liriope, licorice, lonicera, lycium bark, lycium fruit, magnesium, malt, moutan, myrtle. naag bhasma, neem, ophiopogon, oregulin, persica, phaseolus, phellodendron, pine leaves, platycodon, polygonatum, pseudostellaria, pueraria, punir dodi, rehmannia, rose apple, Salacia Oblonga, salvia, saponins, schizandra, scrophularia, setaria (millet), shilajit, sulfur, Swertia Chirayita, tang-kuei, trichosanthes, turmeric, yu-chu, vanadium, and mixtures of all of the specific blood-sugar modulating agents provided thereof.

Bitter gourd nonexclusively includes Momordica dioica, Roxb., and Karela.

Rose apple nonexclusicely includes Eugenia Jambos, Linn., and Jambu Bilva nonexclusively includes Aegle Marmelos, Corr., and Bael fruit.

Neem nonexclusively includes Melia azadirachta, Ravipriya, or Indian Lilac.

The present invention also provides a hair growth composition comprising at least one anti-androgenetic agent and at least one body clock or circadian rhythm normalizing agent.

The body is awash with internal clocks. Researchers know of over one hundred clocks so far (See e.g., Carol Orlock, Inner Time: The Science of Body Clocks and What Makes Us Tick). The human body has inner clocks in nearly every organ, every type of tissue, and inside many cells. All of these clocks have to be synchronized, and all are controlled (and influence) the body's two master clocks, the SCN inside the hypothalamus, and a second unidentified clock that regulates body temperature and alertness.

Chronobiologists divide the clocks into three areas: ultradian rhythms (those shorter than a day); circadian rhythms (24 hour cycles); and infradian rhythms (those cycling in intervals greater than 24 hours). Heart beats, body temperature, breathing patterns, and blink rates are examples of ultradian rhythms. The day/night cycle is circadian. A woman's menstrual cycle is infradian. All of these cycles are governed by hormones released by internal body clocks.

Our seven-day weeks, our calendar year, and our 24 hour days may all seem like inventions of modern society. But they are not. Nature imposes rhythms that are weekly, monthly, and daily on all forms of animal and insect life. We fashioned our time pieces on the model that nature provided for us. How well we perform, how long we endure, our mood, our ability to remember, to feel and express emotions, how well we sense and perceive the world, how alert we are, how safe or accident prone, on and on, all these things are affected by internal clocks.

The expression “body clocks or circadian rhythms” in the present invention nonexclusively includes all clocks in the bodies of living beings, and rhythms such as circadian rhythms, ultradian rhythms, infradian rhythms nonexclusively including circannual rhythms (the annual or yearly cycle used by living things), or circaseptan rhythms (the seven-day cycle in which the biological processes of life, including disease symptoms and development, resolve),

Body clock or circadian rhythm normalizing agents are substances that can help to normalize, regularize, or stabilize a subject's body clocks or circadian rhythms nonexclusively including medicines, amino acids, hormones, and substances nonexclusively including melatonin, amino acids such as 5-Hydroxytryptophan, lithium, melatonin agonists nonexclusively including agomelatine, ramelteon, beta-methyl-6-chloromelatonin, luzindole, and LY156735, herbs or botanicals nonexclusively including German Chamomile, Gotu Kola, Jamaica Dogwood, Kava Kava, Lavender, Lemon Balm, Passionflower, Skullcap, Valerian, and/or mixtures thereof.

Throughout this description, treatment of hair loss includes slowing down, stopping, or reversing hair loss. It also includes indirectly or directly causing hair growth or regrowth. Related conditions nonexclusively include androgenetic alopecia, alopecia areata, anagen effluvium, telogen effluvium and post-partum telogen alopecia, diffuse alopecia, alopecia androgenetica, prostatic hyperplasia, prostatic cancer, hirsutism, acne, male pattern baldness, seborrhea, muscle deterioration, or other diseases not previously mentioned here related to androgen hyperactivity.

Additionally, related conditions nonexclusively include diabetes and other medical disorders such as Attention Deficit Disorder, Depression, and Schizophrenia. Said hair growth composition can also be used for other purposes nonexclusively including building muscle, sharpening mental focus, or as a treatment or form of therapy for varicoceles.

Throughout this patent, terms such as hair growth, treatment of hair loss, and other conditions mentioned above are used interchangeably.

Any of the hair growth compositions described herein can further comprise at least one substance selected from the group consisting of:

(a) at least one blood-sugar modulating agent,

(b) at least one body clock or circadian rhythm normalizing agent,

(c) at least one agent that can reduce or treat side-effects directly or indirectly caused by anti-androgenetic agents (hereinafter “anti-androgenetic side-effect reducing agents”),

(d) at least one agent that can enhance the efficacy of anti-androgenetic agents (hereinafter “anti-androgenetic enhancing agents”), and

(e) omega-3 fatty acids.

For example, a hair growth composition can be comprised of at least one anti-androgenetic agent, at least one blood-sugar modulating agent, and at least one body clock or circadian rhythm normalizing agent.

Anti-androgenetic side-effect reducing agents nonexclusively include one or more of the following:

i. Anti-inflammatory agents nonexclusively including benoxaprofen, centella asiatica, bisabolol, feverfew (whole), feverfew (parthenolide free), green tea extract, green tea concentrate, cat's claw, hydrogen peroxide, lycopene including “Lyc-o-Pen” available from LycoRed Natural Products Industries, Ltd., oat oil, chamomile, and/or mixtures thereof.

ii. Anti-edema agents nonexclusively including bisabolol natural, synthetic bisabolol, and/or mixtures thereof.

iii. Antipruritics and skin protectants nonexclusively including oatmeal, betaglucan, feverfew, soy and derivatives thereof, bicarbonate of soda, colloidal oatmeal, surfactant based colloidal oatmeal cleanser, Anagallis Arvensis, Oenothera Biennis, Verbena Officinalis, and/or mixtures thereof.

As used herein, colloidal oatmeal means the powder resulting from the grinding and further processing of whole oat grain meeting United States Standards for Number 1 or Number 2 oats. The colloidal oatmeal can have a particle size distribution in which not more than 3 percent of the total particles exceed 150 micrometers in size and not more than 20 percent of the total particles exceed 75 micrometers in size. Examples of suitable colloidal oatmeals include, but are not limited to, “Tech-O” available from the Beacon Corporation and colloidal oatmeals available from Quaker.

iv. Collagen enhancers nonexclusively including vitamin A, vitamin C, and/or mixtures thereof.

v. Skin firming agent nonexclusively including dimethylaminoethanol (“DMAE”), and/or mixtures thereof.

vi. Vitamins nonexclusively including the vitamin B complex; including thiamine, nicotinic acid, biotin, pantothenic acid, choline, riboflavin, vitamin B6, vitamin B12, pyridoxine, inositol, carnitine, vitamins A,C,D,E,K and their derivatives such as vitamin A palmitate, vitamin C palmitate, vitamin E acetate, and pro-vitamins, e.g. (i.e. panthenol (pro vitamin B5) and panthenol triacetate) and/or mixtures thereof.

vii. Minerals nonexclusively including selenium, folate, folic acid, zinc, and/or mixtures thereof.

viii. Antibacterial agents nonexclusively including bacitracin, erythromycin, neomycin, tetracycline, chlortetracycline, benzethonium chloride, phenol, and/or mixtures thereof.

ix. Skin emollients and/or skin moisturizers nonexclusively including mineral oil, lanolin, vegetable oils, isostearyl isostearate, glyceryl laurate, methyl gluceth-10, methyl gluceth-20 chitosan, and/or mixtures thereof.

x. Hair conditioners nonexclusively include quaternized compounds such as behenamidopropyl PG-dimonium chloride, tricetylmonium chloride, dihydrogenated tallowamidoethyl hydroxyethylmonium methosulfate, and/or mixtures thereof as well as lipophilic compounds like cetyl alcohol, stearyl alcohol, hydrogenated polydecene, and/or mixtures thereof.

xi. Hair softeners nonexclusively including silicone compounds, such as those that are either non-volatile or volatile and those that are water soluble or water insoluble. Examples of suitable silicones include organo-substituted polysiloxanes, which are either linear or cyclic polymers of monomeric silicone/oxygen monomers and which nonexclusively include cetyl dimethicone; cetyl triethylammonium dimethicone copolyol phthalate; cyclomethicone; dimethicone copolyol; dimethicone copolyol lactate; hydrolyzed soy protein/dimethicone copolyol acetate; silicone quaternium 13; stearalkonium dimethicone copolyol phthalate, stearamidopropyl dimethicone, and/or mixtures thereof.

xii. Hair moisturizers nonexclusively including panthenyl ethyl ether, phytantriol, and/or mixtures thereof.

xiii. Sunscreen agents nonexclusively including benzophenones, bornelone, butyl paba, cinnamidopropyl trimethyl ammonium chloride, disodium distyrylbiphenyl disulfonate, paba, potassium methoxycinnamate, butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, oxybenzone, Padimate O, red petrolatum, and/or mixtures thereof.

xiv. Tanning agents nonexclusively including dihydroxyacetone, and/or mixtures thereof.

xv. Skin lightening agents nonexclusively including hydroquinone, catechol and its derivatives, ascorbic acid and its derivatives, and/or mixtures thereof.

xvi. Insecticides (including insect repellents, anti-scabies and anti-lice treatments) nonexclusively include permethrin, pyrethrin, piperonyl butoxide, imidacloprid, N,N-diethyl toluamide, which refers to the material containing predominantly the meta isomer, i.e., N,N-diethyl-m-toluamide, which is also known as DEET, and/or mixtures thereof.

xvii. Anti fungal agents nonexclusively including tolnaftate, and/or mixtures thereof.

xviii. External analgesics and local anesthetics nonexclusively including benzocaine, dibucaine, benzyl alcohol, camphor, capsaicin, capsicum, capsicum oleoresin, juniper tar, menthol, methyl nicotinate, methyl salicylate, phenol, resorcinol, turpentine oil, and/or mixtures thereof.

xix. Antiperspirants and deodorants nonexclusively including aluminium chlorohydrates, aluminium zirconium chlorohydrates, and/or mixtures thereof.

xx. Counterirritants nonexclusively including camphor, menthol, methyl salicylate, peppermint and clove oils, ichtammol, and/or mixtures thereof.

xxi. Inflammation inhibitors nonexclusively including aspirin, ibuprofen, acetaminophen, cox inhibitors, hydrocortisone, Fragaria Vesca, Matricaria Chamomilla, Salvia Officinalis, and/or mixtures thereof.

xxii. Components that are effective in the treatment of dandruff, seborrheic dermatitis, and psoriasis as well as the symptoms associated therewith. Examples of such suitable benefits agents nonexclusively includes zinc pyrithione, anthralin, shale oil and derivatives thereof such as sulfonated shale oil, selenium sulfide, sulfur; salicylic acid; coal tar; povidone-iodine, imidazoles such as ketoconazole, dichlorophenyl imidazolodioxalan, which is commercially available from Janssen Pharmaceutica, N.V., under the tradename, “Elubiol”, clotrimazole, itraconazole, miconazole, climbazole, tioconazole, sulconazole, butoconazole, fluconazole, miconazole nitrate and any possible stereo isomers and derivatives thereof; piroctone olamine (Octopirox); selenium sulfide; ciclopirox olamine; anti-psoriasis agents such as vitamin D analogs, e.g. calcipotriol, calcitriol, and tacaleitrol; vitamin A analogs such as esters of vitamin A, e.g. vitamin A palmitate, retinoids, retinols, and retinoic acid; corticosteroids such as hydrocortisone, clobetasone, butyrate, clobetasol propionate and/or mixtures thereof.

xxiii. Potassium channel openers or peripheral vasodilators nonexclusively including capsaicin, cayenne, diazoxide, minoxidil, and compounds such as N*-cyano-N-(tert-pentyl)-N′-3-pyridinyl-guanidine (“P-1075”) as disclosed in U.S. Pat. No. 5,244,664, which is incorporated herein by reference, and/or mixtures thereof.

xxiv. Hormones, amino acids, and growth factors nonexclusively including activin, amylin, angiotensin, atrial natriuretic peptide (ANP), calcitonin, calcitonin gene-related peptide, calcitonin N-terminal flanking peptide, ciliary neurotrophic factor (CNTF), corticotropin (adrenocorticotropin hormone, ACTH), corticotropin-releasing factor (CRF or CRH), epidermal growth factor (EGF), insulin growth factor or insulin-like growth factor (IGF), fibroglast growth factor (FGF), follicle-stimulating hormone (FSH), gastrin, gastrin inhibitory peptide (GIP), gastrin-releasing peptide, gonadotropin-releasing factor (GnRF or GNRH), growth hormone releasing factor (GRF, GRH), human chorionic gonadotropin (hCH), inhibin A, inhibin B. insulin, luteinizing hormone (LH), luteinizing hormone-releasing hormone (LHRH), [agr]-melanocyte-stimulating hormone, [bgr]-melanocyte-stimulating hormone, [ggr]-melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, vasoactive intestinal peptide (VIP), vasopressin, the cytokines, colony stimulating factor 4, heparin binding neurotrophic factor (HBNF), interferon-[agr], interferon [agr]-2a, interferon [agr]-2b, interferon [agr]-n3, interferon-[bgr], etc., interleukin-1, interleukin-2, interleukin-3, interleukin-4, interleukin-5, interleukin-6, etc., tumor necrosis factor, tumor necrosis factor-[agr], granuloycte colony-stimulating factor (G-CSF), granulocyte-macrophage, erythropoietin, and/or mixtures thereof.

xxv. Prostaglandins nonexclusively including prostaglandin EI and prostaglandin F2-alpha, and/or mixtures thereof.

xxvi. Heat shock proteins (“HSP”) nonexclusively including HSP 27 and HSP 72, and/or mixtures thereof.

xxvii. Calcium channel blockers nonexclusively including verapamil HCL, nifedipine, diltiazemamiloride, and/or mixtures thereof.

xxviii. Immunosuppressant drugs nonexclusively including cyclosporin and Fk-506, and/or mixtures thereof.

xxix. Transforming growth factor beta.

xxx. Tumor necrosis factor.

xxxi. Non-steroidal anti-inflammatory agents nonexclusively including as benoxaprofen, and/or mixtures thereof.

xxxii. Retinoids nonexclusively including retinoin, and/or mixtures thereof.

xxxiii. Cytokines nonexclusively including IL-6, IL-1 alpha, IL-1 beta, and/or mixtures thereof.

xxxiv. Cell adhesion molecules nonexclusively including ICAM, and/or mixtures thereof.

xxxv. Glucorcorticoids nonexclusively including betametasone, and/or mixtures thereof.

xxxvi. Botanicals and botanical extracts nonexclusively including aloe, clove, ginseng, ginger, horseradish, acai, noni, rehmannia, swertia, sweet orange, zanthoxylum, Serenoa repens (saw palmetto), Hypoxis rooperi, stinging nettle, pumpkin seeds, and rye pollen sandlewood, red beet root, chrysanthemum, rosemary, burdock root, Pygeum Bark Extract, Pygeum africanum, and/or mixtures thereof.

xxxvii. Homeopathic agents such as Kalium Phosphoricum D2, Azadirachta indica D2, Joborandi D1, and/or mixtures thereof.

xxxviii. Genes for cytokines, growth factors, and male-pattered baldness.

xxxix. Antifungals nonexclusively including ketoconazole and elubiol.

xl. Antibiotics nonexclusively including streptomycin.

xli. Protein inhibitors nonexclusively including cycloheximide; acetazolamide; benoxaprofen; cortisone; diltiazem; hexachlorobenzene; hydantoin; nifedipine; penicillamine; phenothaiazines; pinacidil; psoralens, verapamil; zidovudine; alpha-glucosylated rutin having at least one of the following rutins: quercetin, isoquercitrin, hespeddin, naringin, and methylhesperidin, and flavonoids and transglycosidated derivatives thereof which are all disclosed in U.S. patent application No. 7002677 which is incorporated by reference in its entirety herein; and/or mixtures thereof.

xlii. Substances that can enhance sexual functioning nonexclusively including Viagara and Cialis, and/or mixtures thereof.

xliii. Libido enhancing substances such as foods, herbs, and medications nonexclusively including zinc, Wellbutrin, yohimbe, yohimbine, tribulus, maca, caffeine, horny goat weed, and/or mixtures thereof.

xlv. Other cosmetic and pharmaceutical ingredients which are known in the art of pharmacology and cosmetology to treat dermatitis, minor skin irritations, and inhibit inflammation nonexclusively including chamomile extract (matricaria recutitia), cucumber distillate (cucumis sativus), lavender water (lavendula angustifolia), rose water (rosa damascena), witch hazel (hamamelis virginiana), allantoin, bisabolol, rosehip oil, calendula oil, azulaene, menthol, camphor, and/or mixtures thereof.

xlvi. Anti-allergy substances or medications nonexclusively including loratidine, astemizole, montelukast, fexofenadine, other anti-histamines, and/or mixtures thereof. Reducing or treating side-effects indirectly or directly caused by the use of anti-androgenetic agents can be accomplished through mixtures of the above as well.

Anti-androgenetic agent enhancers nonexclusively include one or more of the following:

i. Omega-3 fatty acids.

ii. Polyunsaturated fatty acids, nonexclusively including any of the essential fatty acids (EFA's), such as linoleic and linolenic acid, Gamma-linoleic acid (GLA), eicosapentaenoic acid (EPA), docosahexaenoic acid (DHA), and substances containing said acids nonexclusively including flax oil, sunflower oil, sesame oil coconut oil, evening primrose oil, rice germ and/or oil, bran oil, soy lecithin, oat germ and/or oil, tocotrienols, and/or mixtures thereof.

iii. Omega-9 fatty acids.

iv. Lycopene.

v. Soy and derivatives thereof, nonexclusively including isoflavones.

vi. Allicin.

vii. Quercetin.

viii. Potassium channel openers or peripheral vasodilators nonexclusively including capsaicin, cayenne, diazoxide, minoxidil, and compounds such as N*-cyano-N-(tert-pentyl)-N′-3-pyridinyl-guanidine (“P-1075”) as disclosed in U.S. Pat. No. 5,244,664, which is incorporated herein by reference, and/or mixtures thereof.

ix. Vegetables belonging to the allium category, derivatives thereof, and/or mixtures thereof.

x. Vitamins nonexclusively including the vitamin B complex; including thiamine, nicotinic acid, biotin, pantothenic acid, choline, riboflavin, vitamin B6, vitamin B12, pyridoxine, inositol, camitine, vitamins A,C,D,E,K and their derivatives such as vitamin A palmitate, vitamin C palmitate, vitamin E acetate, and pro-vitamins, e.g. (i.e. panthenol (pro vitamin B5) and panthenol triacetate) and/or mixtures thereof.

xi. Minerals nonexclusively including selenium, magnesium, zinc, and/or mixtures thereof.

xii. Relaxin.

xiii. Botanicals and botanical extracts nonexclusively including aloe, clove, ginseng, ginger, horseradish, acai, noni, rehmannia, swertia, sweet orange, zanthoxylum, Serenoa repens (saw palmetto), Hypoxis rooperi, stinging nettle, pumpkin seeds, and rye pollen sandlewood, red beet root, chrysanthemum, rosemary, burdock root, Pygeum Bark Extract, Pygeum africanum, and/or mixtures thereof.

xiv. Substances that can enhance sexual functioning nonexclusively including Viagara, Cialis, and/or mixtures thereof.

xv. Libido enhancing substances such as foods, herbs, and medications nonexclusively including zinc, Wellbutrin, yohimbe, yohimbine, tribulus, maca, caffeine, horny goat weed, and/or mixtures thereof.

xix. Other hair growth promoter activators which are disclosed in DE 4330597 (as mentioned in U.S. patent application No. 2002035046) which are incorporated by reference in its entirety herein, and/or mixtures thereof.

Enhancing the efficacy of anti-androgenetic agents can be accomplished through mixtures of the above as well.

The present invention also provides a method for the treatment of hair loss comprising providing to a living being an effective amount of any of the hair growth compositions described herein.

The present invention further provides a method for the treatment of hair loss comprising providing to a living being an effective amount of any of the hair growth compositions described herein and practicing at least one of the steps selecting from the group consisting of:

(a) stabilizing the subject's blood sugar;

(b) enhancing blood flow to one or more regions of the subject's body;

(c) preventing the grinding or clenching of the subject's jaw;

(d) consuming low-fat dairy foods;

(e) increasing or maintaining satisfactory folate intake;

(f) sleeping on one or more pillows, pillowcases, or materials resistant to allergens;

(g) engaging in at least one activity proven to treat prostate disorders such as enlargement or cancer;

(h) introducing stem cells into the subject's body.

Stabilizing a subject's blood sugar nonexclusively includes maintaining it at relatively constant levels throughout the day or over a period of time, modulating it, regulating it, reducing it, or narrowing the subject's range of blood-sugar fluctuations throughout the day or over a period of time. It can be achieved through measures nonexclusively including: dieting, exposure to said blood-sugar modulating agents, and/or combinations thereof.

Dieting nonexclusively includes caloric restriction over a period of time, caloric reduction over a period of time, eating frequent yet smaller-than-regular meals throughout the day, eating at a slower pace, eating foods with a relatively low glycemic index, fasting, eating approximately foods from the same food groups approximately around the same times each day, consuming a largely plant-based or vegetarian diet, consuming a diet high in fiber, and/or combinations thereof.

Additional means of stabilizing blood-sugar nonexclusively include: Meditation, relaxation or relaxation treatments, hypnosis, self-hypnosis, various forms of exercise nonexclusively including yoga and/or more strenuous physical activity, acupuncture, biofeedback, hypnosis, self-hypnosis, guided imagery, sound treatments nonexclusively including music or ultrasound, laser treatments, light treatments nonexclusively including exposing the subject to one or more colors or wavelengths, engaging in one or more surgical procedures nonexclusively including transplants such as islet cell transplants, pancreas transplants, or pancreatic cell transplants, and/or combinations thereof.

Enhancing blood flow to a region of the subject's body can be nonexclusively accomplished through: Meditation, relaxation or relaxation treatments, hypnosis, self-hypnosis, various forms of exercise nonexclusively including yoga and/or more strenuous physical activity, acupuncture, biofeedback, hypnosis, self-hypnosis, guided imagery, sound treatments nonexclusively including music or ultrasound, laser treatments, light treatments nonexclusively including exposing the subject to one or more colors or wavelengths, exposure to said potassium channel openers or peripheral vasodilators, and/or combinations thereof.

In cases where hair growth is sought upon a subject's scalp, the subject's scalp, head, neck, face, and/or back regions can be relaxed or targeted to enhance blood-flow to the scalp. Importantly, blood flow enhancing measures can also target one or more of the subject's body parts, or even the subject's body as a whole. For example, certain treatments have been shown to be most effective when applied to the back of a subject's knees.

Preventing the grinding or clenching of the subject's jaw can be accomplished through measures nonexclusively including wearing a bite plate during the day, at night, or both, engaging in the measures to enhance blood-flow provided above, and/or combinations thereof.

Regarding the step of increasing or maintaining adequate folate intake, subjects should preferably ingest levels such as 400 micrograms per day or greater. For certain individuals, less than 400 micrograms per day can suffice. Increasing or maintaining folate intake can be nonexclusively accomplished by eating green vegetables, taking folate or folic acid supplements, eating fortified foods nonexclusively including cereals, and/or mixtures thereof.

Pillows, pillowcases, or materials (hereinafter “pillows”) that are resistant to allergens nonexclusively include pillows comprised of silk. Allergens nonexclusively include pollen, dust mites, and dust mites' waste products. Said pillows can be partially, largely, or entirely resistant to allergens. Subjects can choose to sleep on an entire bed or some other surface or substance that is resistant to allergens as well, and/or combinations thereof.

Engaging in one or more activities proven to treat prostate disorders such as enlargement or cancer nonexclusively includes activities that delay, slow down, treat, or reverse prostate problems. Said problems nonexclusively include prostate enlargement, cancer, and/or combinations thereof. Said activities nonexclusively include surgical procedures, exposure to substances such as medicines, herbs, botanicals, minerals, vitamins, hormones, amino acids, or foods nonexclusively including pomegranates or pomegranate juice, soybeans or soy products, and/or combinations thereof.

Stem cells can be placed, implanted, or grown in a subject's scalp or other parts of his/her body.

In one embodiment, subjects may orally take pill containing at least one anti-androgenetic agent such as finasteride and an alpha-glucosidase inhibitor or a substance such as Salacia oblonga that mimics the effects of alpha-glucosidase inhibitors once per day. In addition, they can also topically apply minoxidil several times per day. In another embodiment, subjects may orally a take pill containing at least one anti-androgenetic agent such as finasteride and at least one agent that normalizes body clocks or circadian rhythms such as melatonin. In addition, they can also topically apply minoxidil several times per day.

In another embodiment, subjects can ingest at least one anti-androgenetic agent at a separate time and frequency from at least one blood-sugar modulating agent and/or at least one agent that normalizes body clocks or circadian rhythms to treat hair loss.

In another embodiment, subjects can ingest a compound comprising: at least one anti-androgenetic agent, at least one anti-androgenetic enhancing agent, and at least one anti-androgenetic side-effect reducing agent, and further engage in at least one blood sugar stabilization activity—such eating numerous small meals each day largely consisting of foods with a low glycemic index or fasting. Said compound can further include at least one body clock or circadian rhythm normalizing agent and/or omega-3 fatty acids.

In another embodiment, subjects can engage in at least one blood-sugar stabilization activity in conjunction with ingesting said hair growth composition.

In another embodiment, subjects can ingest a hair growth composition containing at least one anti-androgenetic agent at the same time, in conjunction, or at a different time (e.g. several times per day, daily, weekly, or monthly) from a separate composition comprising:

(a) at least one blood sugar modulating agent;

(b) at least one agent capable of reducing side-effects indirectly or directly caused by anti-androgenetic agents;

(c) at least one anti-androgenetic enhancing agent;

(d) at least one agent that normalizes at least one of a subject's body clocks or circadian rhythms.

(e) omega-3 fatty acids).

Additionally, to treat hair loss or bring about hair growth, subjects can optionally engage in one or more complementary activities such as:

(a) consuming water at intervals throughout the day;

(b) avoiding foods containing high concentrations of pesticides, herbicides, toxic chemicals, and/or antibiotics;

(c) increasing or maintaining adequate sun exposure;

(d) ingesting at least the RDA recommended amount of Vitamin D per day;

(e) ingesting sprouts;

(f) controlling addictive behaviors;

(g) getting a satisfactory amount of sleep;

(h) engaging in at least one activity that can normalize a subject's body clock and/or circadian rhythms;

(i) protecting the scalp from the sun's radiation;

Avoiding foods with high concentrations of pesticides and/or antibiotics can be nonexclusively accomplished by consuming a diet containing bioengineered foods or foods that have been certified as organic, or mixtures thereof.

Sprouts nonexclusively include vegetable sprouts such as broccoli sprouts, grain sprouts such as wheat sprouts, bean sprouts such as mung bean sprouts, herbal sprouts such as mustard sprouts, and/or mixtures thereof.

Controlling addictive behaviors includes decreasing their frequency or stopping them. Controlling addictive behaviors can be nonexclusively accomplished by abstaining or withdrawing from the sources of the addiction or undergoing treatments for the addictive behaviors nonexclusively including 12 step programs or other similar programs, and/or combinations thereof.

Engaging in one or more activities that can normalize a subject's body clock(s) and/or circadian rhythms nonexclusively includes measures such as exposing the subject to said body clock or circadian rhythm normalizing agents, going to sleep and waking up at approximately the same times each day, exposing a subject to sensory compositions (such as music or light shows) derived from their own brain waves that were previously recorded at specific times during the day (such as the service offered by Brain Music Therapy in New York City), sufficient exposure to natural light sources or artificial light sources (hereinafter “sufficient light sources”) nonexclusively including light boxes or light bulbs emitting full-spectrum light such as bulbs manufactured by Ott-Lite, transcranial magnetic stimulation, vagus nerve stimulation, accupunture, and/or combinations thereof. Normalizing a subject's body clock or circadian rhythms nonexclusively includes stabilizing or regularizing them.

Protecting the scalp from the sun's radiation involves taking measures to prevent the sun's radiation from harming follicles in the scalp area. Said measures nonexclusively include wearing a cap or a hat, applying sunscreen, and/or combinations thereof.

According to the present invention, there is provided a composition which can be applied topically in the form of lotion, ointment, gel, cream, patch, or systemically for internal or parenteral use in the form of capsules, tablets, or ampules, or other means of delivery (nonexclusively including inhalation, injections, snorting, swallowing, and/or smoking) for the treatment of hair loss and related conditions.

In one embodiment, said compositions can be in the form of creams, lotions, ointments, gels, or patches, prepared for use in any conventional manner, in admixture with one or more physiologically acceptable carriers and diluents.

Said compositions may take such forms as suspension, solutions, or emulsions in oily or aqueous vehicles, and may contain agents such as emulsifying, suspending, stabilizing, gelling and/or dispersing agents. Alternatively, the active ingredients may be in powder form for constitution with a suitable vehicle (i.e., sterile, pyrogen-free water) before use and may be compounded into tablet, powder or capsule form using techniques well known to those skilled in the formulary art.

The formulations of the present invention comprise at least one active ingredient, as above defined, together with one or more acceptable carriers and optionally other therapeutic ingredients. The carrier(s) should be acceptable in the sense of being compatible with the other ingredients of the formulation and not deleterious to the recipient thereof.

An effective amount of the active ingredient in said hair growth composition means an amount effective for treating hair loss or growing hair, and preferably may range from, based upon the total weight of said hair growth composition, from about 0.001 percent to about 20 percent, and preferably from about 1 percent to about 5 percent.

The formulations may conveniently be presented in unit dosage form and may be prepared by any of the methods well known in the art of pharmacy. Such methods include the step of bringing into association the active ingredient with the carrier, which constitutes one or more accessory ingredients. Formulations may be prepared by uniformly and intimately bringing into association one or more active ingredients with liquid carriers or finely divided solid carriers, or both, and then, if necessary, shaping the product.

Some formulations can applied as a topical lotion, ointment, gel or cream, containing the active ingredient in a concentration of, for example, 0.005 to 10.0 percent. When formulated in cream, the active ingredients may be employed with an oil-in-water cream base.

If desired, the aqueous phase of the cream base may include, for example, at least thirty percent (30%) w/w of a polyhydric alcohol (i.e., an alcohol having two or more hydroxyl groups such as propylene glycol, butane-1,3-diol, mannitol, sorbitol, glycerol and polyethylene glycol and/or mixtures thereof. The topical formulations many desirably include a compound which enhances absorption or penetration of the active ingredient through the skin or other affected areas. Examples of such dermal penetration enhancers include dimethylsulphoxide and related analogues.

The oily phase of the emulsions of this invention may be constituted from known ingredients in a known manner. The oil phase may comprise merely an emulsifier it desirably comprises a mixture of at least one emulsifier with a fat or an oil or with both a fat and an oil. Preferably, a hydrophilic emulsifier is included together with a lipophilic emulsifier, which acts as a stabilizer. It is also preferred to include both an oil and a fat. Together, the emulsifier(s), with or without stabilizers(s), make up the so-called emulsifying wax, and the wax, together with the oil and/or fat, make up the so-called emulsifying ointment base, which forms the oily dispersed phase of the cream formulations.

Emulgents and emulsion stabilizers suitable for use in the formulation of the present invention include Tween 60, Span 80, cetostearyl alcohol, myristyl alcohol, glycerl mono-stearate and sodium lauryl sulphate.

The choice of suitable oils or fats for the formulation is based on achieving the desired cosmetic properties, because the solubility of the active compound in most oils likely to be used in pharmaceutical emulsion formulations is very low. Therefore, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester or coconut fatty acids, isopropyl myristate, decyl oleate, isopropyl palmitate, butyl stearate, 2-ethylhexyl palmitate, or a blend of branched chain esters known as Crodamol CAP may be used, the last three being preferred esters. These may be used alone or in combination, depending on the properties required. Alternatively, high melting-point lipids, such as white soft paraffin and/or liquid paraffin, or other mineral oils, can be used.

The present invention can be applied to all living beings nonexclusively including humans, dogs, cats, and other pets such as guinea pigs.

While the invention will now be described in connection with certain preferred embodiments in the following examples so that aspects thereof may be more fully understood and appreciated, it is not intended to limit the invention to these particular embodiments. On the contrary, it is intended to cover alternatives, modifications and equivalents as may be included within the scope of the invention as defined by the claims. Thus, the following examples, which include preferred embodiments, will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

### EXAMPLE 1

A user (hereinafter interchangeable with “subject”) wakes up in the morning and takes a pill containing Finasteride and Byetta.

### EXAMPLE 2

The user ingests a pill containing Finasteride, Minoxidil, lycopene, isoflavones, capsaicin, zinc, omega-3 fatty acids, fiber, Salacia oblonga, and cinnamon several times per day.

### EXAMPLE 3

A user (hereinafter interchangeable with “subject”) wakes up in the morning and takes a pill containing Finasteride and Byetta. The user then takes a shower. During the shower, the user shampoos his (or her) hair with Pentrax, a shampoo containing coal tar (5%). After showering and drying off his hair, the user applies Rogaine (minoxidil) to his scalp area. The user then eats the first of 5-8 small meals during the day.

At this first meal, the user eats a piece of organic fruit, oatmeal, and some Brazil nuts. After each meal, the user takes a cinnamon extract pill to slow his body's conversion of the starches in his diet to sugar. The user supplements this meal with 2 teaspoon-full servings of flax-seed oil. The user then goes to work.

Throughout the day, the user drinks water on a regular basis. The user also eats two or more small meals during the workday largely composed of foods with a relatively low glycemic index such as beans instead of one big lunch. If the user snacks during the day, the user largely refrains from foods that are high in refined grains or added sugars such as candy bars, opting instead for raw vegetables such as celery sticks. Additionally, the user also takes one or more 10-15 minute breaks to walk around, stretch out his neck, and to breathe deeply or meditate to relax.

After work, the user stops by the gym to exercise and to stretch further.

Once home, the user can have two or more small meals before going to sleep. The user may choose to eat a serving cold-water fish such as salmon at one of these meals. At another meal, the user may eat a whole-grain pasta dish containing plum tomatoes (which are higher in lycopene content than other tomatoes), tofu, spicy peppers, ginger, garlic, shallots, and horseradish.

Before going to sleep, the user again applies Rogaine to his scalp area.

The above steps can be repeated on a daily basis.

### EXAMPLE 4

The user ingests a pill containing finasteride, lycopene, and an agent that can normalize one of the subject's body clocks one hour before going to sleep each night.

### EXAMPLE 5

Each morning, upon waking up, the user reads the newspaper seated in front of a light box for 30 minutes. The user then washes up and applies an ointment to both his scalp and the back of his knees containing duasteride, lycopene, isoflavones, zinc, omega-3 fatty acids, melatonin, and bitter melon. The user applies said ointment later in the day as well. The user wears a bite plate both at work and later while sleeping to address his jaw-grinding problem.

The user gets two massages per week. The user also avoids eating pesticide-laden fruits and vegetables at conventional supermarkets, opting instead to eat fruits and vegetables purchased either from an organic supermarket or from directly farmers growing bioengineered strains developed by a company such as Monsanto.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

What is claimed is:

1. A hair growth composition comprising:
(a) at least one anti-androgenetic agent, and
(b) at least one blood-sugar modulating agent.

2. A hair growth composition of claim 1 wherein the blood-sugar modulating agent is selected from the group consisting of:
(insulin, sulfonylureas, biguanides, meglitinides, prandial glucose regulators, alpha-glucosidase inhibitors, thiazolidinediones, glitazones, acarbose, aspirin, hormones that are naturally occurring in humans or animals, or synthetic versions, Byetta, Symlin, aconite, adenophora, alisma, amalaki churna, anemarrhena, asparagus root, astragalus, atractylodes, banaba plant, benincasa, bitter gourd, bitter melon, bilva, carthamus, chandraprabhavati, chromium, chrysanthemum, cinnamon, clerodendron, codonopsis,: coix, coptis, cornus, corn silk, cumin, cuscuta, cyperus, Dakhni Mirch, dandelion, dendrobium, dioscorea, elecampane, epimedium, eriocaulon, fenugreek, fo-ti, furmar, ginseng, glehnia, glucomannan, gymnema, gypsum, ho-shou-wu, hoelen, jambu, jamun-ki-guthali, juniper berries, konjac, laminaria, liriope, licorice, lonicera, lycium bark, lycium fruit, magnesium, malt, moutan, myrtle, naag bhasma, neem, ophiopogon, oregulin, persica, phaseolus, phellodendron, pine leaves, platycodon, polygonatum, pseudostellaria, pueraria, punir dodi, rehmannia, rose apple, Salacia Oblonga, salvia, saponins, schizandra, scrophularia, setaria [millet], shilajit, sulfur, Swertia Chirayita, tang-kuei, trichosanthes, turmeric, yu-chu, vanadium).

3. A hair growth composition comprising:
(a) at least one anti-androgenetic agent, and
(b) at least one agent that can normalize at least one of a subject's body clocks or circadian rhythms.

4. A hair growth composition comprising:
(a) at least one anti-androgenetic agent, and
(b) at least one agent that can normalize at least one of a subject's body clocks or circadian rhythms other than melatonin.

5. A hair growth composition of claim 1 or 3 further comprising at least one substance selected from the group consisting of:
(a) at least one blood-sugar modulating agent;
(b) at least one agent that can normalize at least one of a subject's body clocks or circadian rhythms;
(c) at least one anti-androgenetic side-effect reducing agent;
(d) at least one anti-androgenetic enhancing agent;
(e) omega-3 fatty acids).

6. A method for the treatment of hair loss comprising:
providing to a subject an effective amount of the hair growth composition of claim 1, 2, 3, 4, or 5.

7. The method of claim 6 further comprising at least one step selected from the group consisting of:
(a) stabilizing the subject's blood sugar;
(b) enhancing blood flow to one or more regions of the subject's body;
(c) preventing the grinding or clenching of the subject's jaw;
(d) consuming low-fat dairy foods;
(e) increasing or maintaining satisfactory folate intake;
(f) sleeping on one or more pillows, pillowcases, or materials resistant to allergens;
(g) engaging in at least one activity proven to treat prostate disorders such as enlargement or cancer;
(h) introducing stem cells into the subject's body.)

8. The hair growth composition of claim 1 or 3 further comprising a pharmaceutically acceptable carrier.

9. The hair growth composition of claim 8 wherein said pharmaceutically acceptable carrier is an oral dosage form.

10. The hair growth composition of claim 8 wherein said pharmaceutically acceptable carrier is a topical carrier.

11. The hair growth composition of claim 1 or 3 wherein the composition is in a form selected from the group consisting of: (a food, pill, cream, lotion, ointment, patch, or vapor).

12. The method of claim 11, further comprising the step of applying the hair growth composition of claim 1 or 3 to at least one portion of a body.

13. A method for the treatment of hair loss comprising:
(a) providing a subject with an effective amount of an anti-androgenetic agent;
(b) providing a subject with an effective amount of at least one substance selected from the group consisting of:
(i) at least one blood-sugar modulating agent;
(ii) at least one agent that can normalize at least one of a subject's body clocks or circadian rhythms;
(iii) at least one anti-androgenetic side-effect reducing agent;
(iv) at least one anti-androgenetic enhancing agent;
(v) omega-3 fatty acids);
(c) engaging in at least one step selected from the group consisting of:
(i) stabilizing the subject's blood sugar;
(ii) enhancing blood flow to one or more regions of the subject's body;
(iii) preventing the grinding or clenching of the subject's jaw;
(iv) consuming low-fat dairy foods;
(v) increasing or maintaining satisfactory folate intake;
(vi) sleeping on one or more pillows, pillowcases, or materials resistant to allergens;
(vii) engaging in at least one activity proven to treat prostate disorders such as enlargement or cancer;
(viii) introducing stem cells into the subject's body).
